(19) United States
(12) Patent Application Publication
(10) Pub. No.: US 2013/0248598 A1
(43) Pub. Date: Sep. 26, 2013

(54) Payload Aware Medical Cart, System and Method

(71) Applicant: Pourya Dehnadi, Incline Village, NV (US)

(72) Inventor: Pourya Dehnadi, Incline Village, NV (US)

(21) Appl. No.: 13/899,817

(22) Filed: May 22, 2013

Related U.S. Application Data
(63) Continuation of application No. 12/429,873, filed on Apr. 24, 2009, now abandoned.

(51) Int. Cl. G06Q 10/08; G06Q 50/22

(52) U.S. Cl. CPC G06Q 10/087; G06Q 50/22
USPC 235/385

(57) ABSTRACT

A payload aware medical cart, system and method utilizes a computer system to take inventory of the medical products stored in the medical cart. Each medical product carries an ID tag that provides a unique identifier when queried by an ID sensor, and the medical cart detects the contents of each compartment by reading the ID tags of the products placed in that compartment thereby producing an inventory enumerating all products and the quantity of each product per compartment.

### CROSS-REFERENCE TO RELATED PATENT APPLICATION

This application claims priority from prior provisional U.S. patent application Ser. No. 61/125,317 filed Apr. 24, 2008, the entire disclosure of which is incorporated herein by reference.

### BACKGROUND OF THE INVENTION

This invention generally relates to medical carts storing medical products for patient care and, more particularly, to such carts for use in medical procedures in, for example, hospitals, clinics and emergency situations where the carts are aware of their payload contents and systems and methods employing such carts.

Typical medical carts provide the products necessary to perform particular medical procedures, such as in the event of an emergency for example, or products required for other situations. For example, hospitals generally require that the contents of a crash cart follow a policy where each compartment of the cart is identified and its contents are listed with respect to product and quantity. Once a cart is fully stocked, it is locked and stored in an accessible location so that it is ready to be used in an emergency situation. Far too often, it is the case that once the cart is opened under duress, the necessary contents are either misplaced into the wrong compartment, or missing altogether.

Currently, medical carts stocked with supplies and equipment (medical products) are manually checked to determine whether they contain the products required by hospital policy. The act of manually checking carts generally involves a person taking inventory by visually inspecting the contents of a cart. The inventory is compared to a list prescribing the products that the cart is required to contain which is referred to as the Cart Configuration Policy (CCP). If the cart is missing any medical products listed on the CCP, the inspector must obtain the missing products and stock the cart accordingly.

Medical carts typically contain multiple compartments that carry the medical products (payload). Compartments are generally drawers or cabinets, but may take other forms. The CCP typically describes the location of the contents with respect to specific compartments. Therefore, it is necessary for the inspector to also ensure that the products are placed in the correct compartment.

The CCP may be developed and potentially mandated by the hospital, but it can also be part of a standard. For example, the Advanced Cardiac Life Support (ACLS) prescribes algorithms to be used when a patient is perceived to have stopped breathing and/or is experiencing cardiac arrest. This is commonly referred to as “Code Blue.” Such an algorithm involves supplies and equipment (medical products) that are necessary for the medical practitioner to perform the procedures in order to attempt to save a person's life. For that reason, it is imperative that carts be prepared and ready for such events which may precipitate suddenly. Hospitals following the ACLS algorithm take inventory of the cart as described above and lock the cart to ensure that supplies or equipment are not removed from the cart for routine or other use. Under a “Code Blue” situation, the cart is rushed to the scene and unlocked for access to its contents. Once the cart has been used, it must be restocked and locked for later use.

Often, due to human error, the cart is not adequately configured according to the CCP for intended use. The range of implications of such an error could be from mere inconvenience to potentially life threatening depending on the type of cart that has been incorrectly stocked.

In order to reduce errors, hospitals generally mandate policies requiring carts to be checked periodically and sometimes require clipboards or notebooks to be attached to the cart to record the last time the cart was checked and by whom. However, mistakes still occur, and those mistakes can be costly.

In view of the above, there is a need to remedy inadequately configured carts, i.e. for a cart to be aware of the contents in each of its compartments, that is, to be “payload aware.”

### SUMMARY OF THE INVENTION

In one aspect, the present invention is embodied in a medical cart having a plurality of compartments for storing medical products carrying identification tags, sensors in the compartments for sensing the medical product identification tags to detect the presence of the medical products in the compartments and providing detection signals representative of the detected products and a processor carried by the cart receiving the detection signals and providing an indication of the medical products carried by the cart.

In another aspect, the medical cart of the present invention has a plurality of compartments and a sensor in each compartment such that the medical products in each compartment can be identified to provide data to a processor carried by the cart.

In a further aspect, the present invention is embodied in a system and method using a payload aware medical cart including a processor receiving data indicating the medical products carried and comparing the data with the data representing a cart configuration policy to provide an indication of any medical products missing from the medical cart.

The present invention includes a payload aware medical cart, system and method utilizing a computer system to take inventory of the contents of the cart. The inventory process enumerates all products and the quantity of each product per compartment. The result of the findings is compared to the cart's configuration policy (CCP). Each item being placed in the payload cart carries an ID tag that provides a unique identifier when queried by a sensor. The cart detects the contents of each compartment by reading the ID tags of the products placed in that compartment. Each compartment can be equipped with its own ID reader, or a central ID reader can be carried by the cart at a location to receive signals from an antenna in each compartment. Each compartment can be shielded such that the contents of neighboring compartments or nearby carts are not detected and mistakenly accounted for in the inventory. The ID reader or readers are connected to a central processing unit (CPU) that is carried by the cart. The CPU compiles the current state of the cart by querying the contents of each compartment and aggregating the results. The cart notifies appropriate personnel or a central computer as to the compliance state of the cart.

Accordingly, aspects and advantages of the present invention include significantly reducing or altogether eliminating human error when stocking medical carts, increasing the efficiency with which medical carts are checked for compliance with the CCP, making medical carts aware of their CCP so that the medical cart itself knows what products it contains, what compartments should contain particular products and whether the medical cart complies with the prescribed CCP enabling the medical cart to place orders automatically with a central supply distribution service, such as in a hospital, for missing products enabling the medical cart to guide medical staff in configuring itself properly, in the case that a medical cart does not have a prescribed CCP, the medical cart can report its inventory, and providing a payload aware cart where each product that is placed into a compartment is identified by a type of tag that can be easily detected without human intervention, examples of such tags being RFID (radio frequency identification) and USID (ultrasonic identification). RFID tags can be passive or active; however, it is more practical to utilize a passive RFID tag. Each designated compartment has its own ID reader or antenna (sensor) that detects the presence of products that have been identified by the ID tags. Each sensor is connected to a processor (microcontroller) carried by the medical cart that performs central processing.

Other objects and advantages of the present invention will become apparent from the following description of the preferred embodiments taken in conjunction with the accompanying drawings, wherein like parts in each of the several figures are identified by the same reference characters.

### BRIEF DESCRIPTION OF THE DRAWINGS

FIG. 1 shows a payload aware medical cart according to the present invention.

FIG. 2 is a block diagram of a payload aware medical cart according to the present invention.

FIG. 3 is a flow chart illustrating operation of the payload aware medical cart system and method of the present invention.

FIG. 4 is a flow chart illustrating the entering of cart configuration by direct input into server software.

FIG. 5 is a flow chart illustrating entering of cart configuration by recording a cart's current inventory as a cart configuration policy.

FIG. 6 is a flow chart illustrating initiation of a compliance check and reporting results.

FIG. 7 is a flow chart illustrating stocking of a non-compliant cart.

### DESCRIPTION OF THE INVENTION

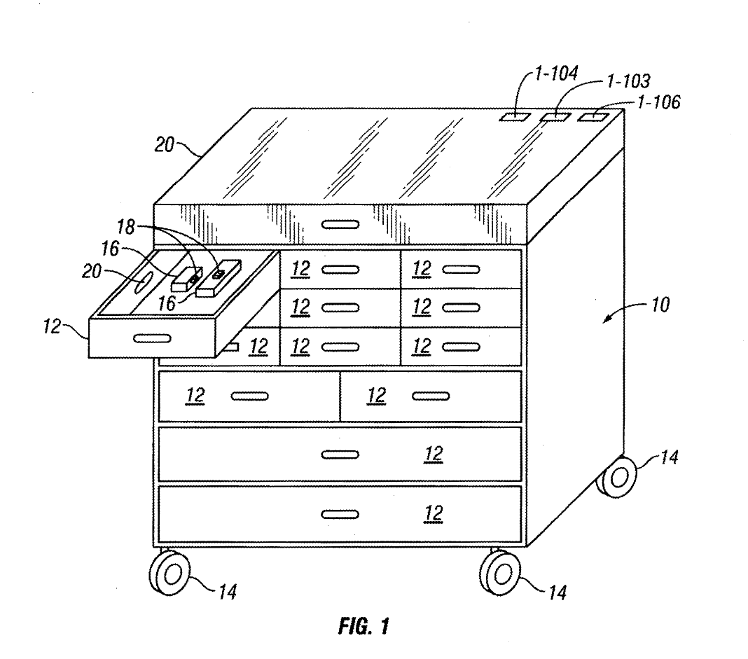

A payload aware medical cart 10 according to the present invention is shown in FIG. 1 and includes a plurality of designated compartments 12. The compartments are generally drawers or cabinets, but other compartment types can be used as in conventional medical carts. There are many types and configurations of medical carts with which the present invention can be used as will be appreciated from the Background of the Invention and as evidenced by, for example, the medical carts available from Armstrong Medical Industries, Inc. and Lionville Systems, Inc. Many currently available medical carts have particular designations such as, for example, “code blue carts” described above and “code carts” and crash carts” which are used in emergency situations. The medical cart 10 is preferably mounted on wheels 14 or rollers to facilitate movement of the medical cart to various locations in a medical facility such as a hospital. Medical products 16 are stored in the compartments 12 and carry identification tags 18 of any type capable of being sensed. The identification tags 18 will be referred to hereinafter as RFID; however, it should be understood that, while RFID tags are preferred from a current practical and cost effective standpoint, it is anticipated that advances in technology will allow the use of other types of identification tags with the present invention. Sensors 20 are disposed in each compartment 12 for sensing the medical products 16 via the identification tags 18 to detect the presence of the medical products in the compartment and provide detection signals to a processor (CPU) carried by the medical cart, for example in compartment 20 on the top of the medical cart.

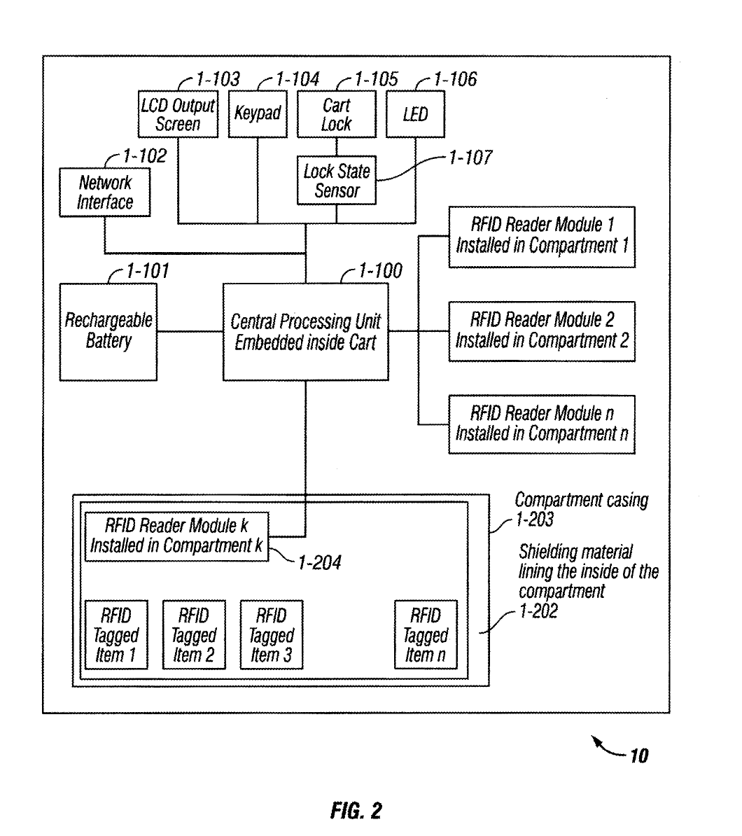

As shown in FIG. 2, the medical cart 10 contains a central processing unit (CPU) 1-100 that is connected to a rechargeable battery 1-101 to draw power for operation. The CPU (processor) may be network enabled by connection to a network interface 1-102, such as Ethernet, or wireless protocols such as IEEE 802.11a/b/g/n or Wi-Fi. The medical cart 10 contains a cart lock mechanism 1-105 connected to a lock state sensor 1-107 which is connected to the CPU. The lock state sensor reports to the CPU whether the cart lock is locked or unlocked. The cart may provide for user input and/or output. For input, a keypad 1-104 is provided. For output, an LCD output screen 1-103 and/or one or more LEDs 1-106 is provided. The input and output devices are preferably disposed on the medical cart at accessible locations to enable a user to interact with the CPU.

Each medical cart compartment 12 contains a sensor in the form of a RFID reader module 1-204 in the embodiment shown in FIG. 2. The sensor can take the form of an antenna supplying detection signals to a central RFID reader using multiplexing technology. Each compartment 12 can be shielded using a shielding material 1-202 disposed within the compartment casing 1-203, such as aluminum or other substances capable of blocking radio waves. Shielding prevents the RFID reader module from detecting any RFID tags outside of the compartment in which the reader module (sensor) is installed. Each RFID reader module is connected to the CPU either via wires or wirelessly and is thus able to communicate with the CPU. The cart can have as many RFID reader modules as it has compartments or a single reader as noted above.

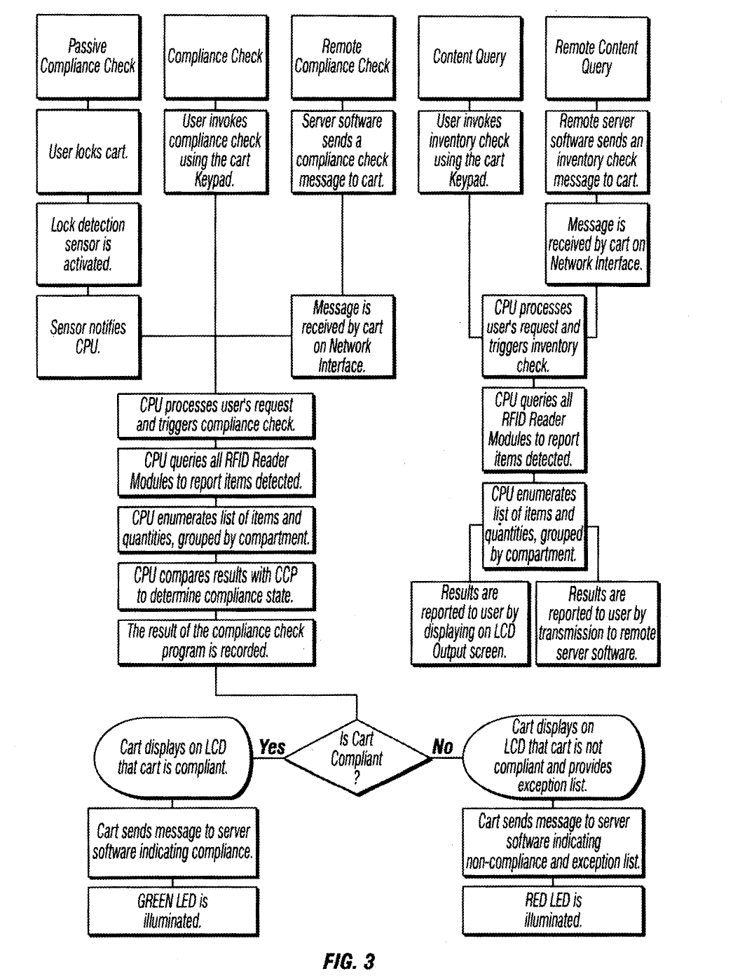

In operation, the medical cart performs the functions of taking inventory of the contents of the medical cart, reporting the results and comparing the inventory of the medical cart to a cart configuration policy (CCP). Modes of operation of the system and method of the present invention using medical cart 10 are shown in FIG. 3 and include a content query mode invoked by a user by enabling the user input controls to determine the cart contents. In a compliance check mode, the medical cart contents are compared to a CCP. The medical cart indicates a compliant state or a non-compliant state depending on whether the contents (medical products) match the CCP or not, respectively. This mode requires no network connectivity, no remote computer system (i.e. PC), and no ancillary software installed on the remote computer system. In a remote content query mode, the contents of the medical cart are remotely queried by the server software. This mode requires no CCP but does require network connectivity to a remote computer system that runs ancillary software capable of communicating with the medical cart. In a remote compliance check mode, the compliance check mode above is invoked remotely from the server software. This mode requires the CCP and network connectivity to a remote computer system that runs ancillary software capable of communicating with the cart. In a passive compliance check mode, the compliance check mode above is invoked by default based on user action, such as the locking of the cart.

In the case of the content query mode and the remote content query mode, when the medical cart is invoked it performs the inventory check and reports the results to the user. The results can be reported in a number of ways, such as using the onboard LCD output screen; or, in the case of the remote content query mode, the results are transmitted via the network interface to a computer capable of displaying those results. This is the default operation of the medical cart when the medical cart has no CCP loaded.

When the medical cart has a CCP loaded, it still performs the inventory check, but it also performs a compliance check. A compliance check is a comparison of the results of the inventory check against the CCP. If the inventory results of the medical cart match the CCP, the medical cart is deemed to be compliant. However, if the inventory results of the cart do not match the CCP, then the cart is deemed to be non-compliant. Compliance check can be invoked using the modes described above, i.e. compliance check, remote compliance check, and passive compliance check mode

To perform a compliance check, it is first necessary to load the CCP so that it is accessible by the medical's cart's CPU. One method to enter the CCP is to input the CCP data directly into the medical cart using the medical cart's keypad input.

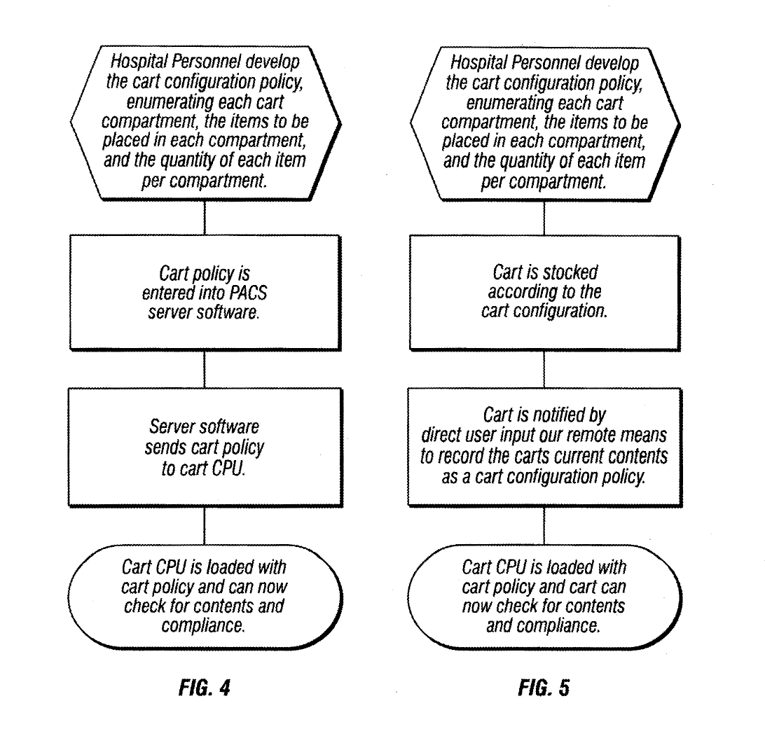

Another method is shown in FIG. 4 where the CCP is entered into compatible software that runs on a remote computer. The remote computer can then transmit the CCP to the medical cart. This method requires designated personnel to develop the requirements of the CCP which is entered into compatible software. Once completed and satisfied, the designated personnel will invoke the software to send the CCP to the medical cart CPU. The medical cart CPU will thus be loaded with a CCP that it can use to check its inventory against and report compliance.

Alternatively, as shown in FIG. 5, the CCP is entered by simply capturing the state of the medical cart upon stocking it to match a prescribed configuration. Similar to the method described above in FIG. 4, designated personnel will develop the requirements of the CCP. Instead of entering it into the software, the designated personnel will stock the medical cart as it should be in a compliant state. The medical cart is then notified by direct user input or remote invocation to record the medical cart's contents as a CCP. The medical cart CPU is thus loaded with a CCP that it can use to check its inventory to report its compliance state.

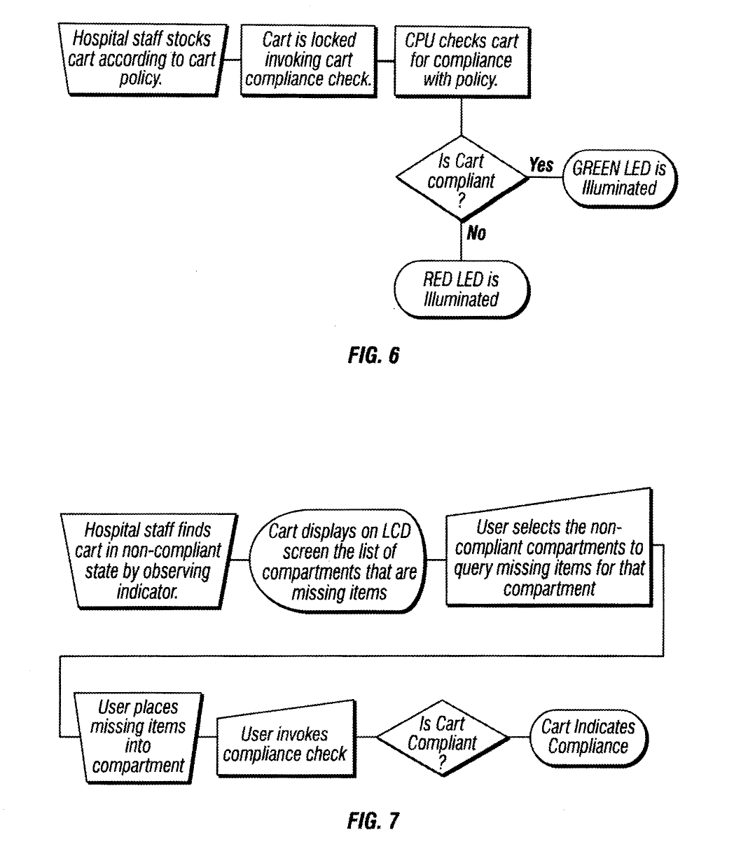

Once the medical cart has a CCP loaded, a user can invoke the medical cart to perform a compliance check. Invoking compliance check is generally performed by locking the medical cart to commence a passive compliance check. Once a medical cart is locked, the lock state sensor notifies the CPU that the medical cart lock is locked. The CPU then performs the compliance check. An example of this method is shown in FIG. 6 where hospital staff stock the medical cart according to a particular policy. Then, the medical cart is locked invoking a compliance check. The CPU checks for compliance against the loaded CCP. If the medical cart is compliant, the medical cart illuminates an LED to the color green. If it is non-compliant, it illuminates an LED to the color red. Alternatively, the medical cart may indicate compliance state by illuminating separate LED's, outputting a message to the LCD output display, or generating an audible sound to indicate state. These alternate means of indicating compliance state provide users with sight and/or hearing deficiencies, as well as color-blindness, accessibility to using the medical cart. When connected to a network, the compliance state can be reported to ancillary software running on a remote computer. The software can, in turn, perform a number of remote notifications, such as posting the state to a user interface on a remote system, such as a web site or sending an email, a text message (SMS), or other form of electronic notification to designated personnel informing them of the state of the medical cart.

in the event that a medical cart is non-compliant, as shown in FIG. 7, the medical cart will communicate its state to hospital staff or other personnel by triggering an indicator as described above. The hospital staff observes the indicator and proceeds to query the medical cart for its missing medical products. This can be done by using the LCD output display, or using the remote software. The user then determines which items are missing, in what quantities, and from which compartments of the medical cart since the sensors in each compartment permit the processor to determine content compliance in each compartment. The user then places the missing medical products into the appropriate compartments. Once placed, the user invokes a compliance check. The CPU performs the compliance check and once again indicates the medical cart's compliance state. If the medical cart continues to be non-compliant then these steps can be repeated to bring the cart to compliance. Alternatively, non-compliance may be the result of erroneous information in the CCP. The user may determine that the CCP requires modification and may wish to alter the CCP and upload a new CCP and re-invoke the compliance check.

Since the CCP should not be freely altered, security, such as password protection, is beneficial to avoid tampering with the CCP.

The medical cart can be loaded with as many CCP's as it has memory to store. Each CCP can be represented by a unique identifier. The users of the medical cart can select the CCP they choose to be active. The active CCP is the CCP that the medical cart will use to perform a compliance check. To switch between CCPs, the user selects another CCP to become the active CCP. The medical cart should have only one active CCP.

The present invention, thus, provides a payload aware medical cart having a plurality of compartments for receiving medical products for use in a particular medical procedure or situation carrying ID tags, a sensor in each compartment and a processor carried by the medical cart for receiving detection signals from the sensors for determining medical product inventory, a system using the payload aware medical cart for determining medical cart compliance and what medical products are missing, and a method using the payload aware medical cart to determine and assure medical cart and compartment compliance.

As previously noted, an alternative to the payload aware medical cart system described above can be implemented by placing one reader (or more) centrally, such as at the medical cart's CPU, and connecting the antennae in each compartment to a multiplexer such that a single reader can monitor multiple antennae. For example, SkyeTek makes SkyePlus multiplexers that can connect 4 or 8 antennas to a single SkyeModule reader. This design significantly reduces the number of readers needed and, therefore, reduces cost per medical cart. Instead of RFID, Ultrasonic Identification (USID) can be used as the enabling technology. USID has the advantage of not interfering with systems susceptible to radio interference, but it does not have widespread usage and cost per tag is prohibitive. However, in the event that USID becomes prevalent, the medical cart can use USID readers to detect the contents of the compartments. USID can also be shielded so that the signal stays within a compartment thereby minimizing the risk of erroneously reading a neighboring compartment or medical cart's contents. The medical cart should ideally be constructed from material that has the least impact on MRI equipment, such as aluminum and plastics rather than steel.

“Medical products” as used herein includes equipment, instruments, tubing, syringes, catheters, pharmaceuticals, wipes and all items used in medical procedures, treatments and situations. The identification tags can be of any type to permit sensing thereof and can be carried externally or internally of the medical products or the packaging therefor. The sensors can be any type of device capable of sensing the identification tags and providing detection signals indicating the presence of medical products in a designated compartment.

Inasmuch as the present invention is subject to many variations, modifications and changes in detail, it is intended that all subject matter discussed above or shown in the accompanying drawings be interpreted as illustrative only and not be taken in a limiting sense.

What is claimed is:

1. A payload aware medical cart comprising
a medical cart having a plurality of designated compartments for storing medical products carrying identification tags;
sensor means in said designated compartments for sensing said medical product identification tags to detect the presence of said medical products in said designated compartments and providing detection signals representative of said detected medical products; and
processor means carried by said medical cart for receiving said detection signals and generating an inventory output indicating said medical products carried by said medical cart.

2. The payload aware medical cart recited in claim 1 wherein said processor means generates inventory outputs indicating said medical products in each of said designated compartments separately.

3. The payload aware medical cart recited in claim 1 wherein said identification tags are RFID units.

4. The payload aware medical cart recited in claim 1 wherein said identification tags are USID units.

5. The payload aware medical cart recited in claim 1 wherein said sensor means in each of said designated compartments includes an antenna for sending identification data to said processor means and said processor means reads said identification data to identify each medical product in a designated compartment.

6. The payload aware medical cart recited in claim 1 wherein said sensor means in each of said designated compartments includes a reader for identifying each medical product in a designated compartment.

7. The payload aware medical cart recited in claim 1 wherein said designated compartments are lined with shielding material.

8. A payload aware medical cart system comprising
a medical cart having a plurality of designated compartments for storing medical products carrying identification tags, sensor means in said designated compartments for sensing said medical product identification tags to detect the presence of said medical products in said designated compartments and providing detection signals representative of said detected medical products, and processor means carded by said medical cart for receiving said detection signals and generating an inventory output indicating said medical products carried by said medical cart;
an input device for supplying said processor means with a cart configuration policy listing of medical products;
means for comparing said inventory output with said cart configuration policy listing of medical products; and
output means providing an indication of whether said medical cart carries an inventory of medical products to be compliant with said cart configuration policy listing of medical products.

9. The payload aware medical cart system recited in claim 8 wherein said cart configuration policy listing of medical products includes a listing of medical products for each designated compartment and said output means provides an indication of whether a designated compartment stores the medical products listed for said designated compartments.

10. A method of managing the storage of medical products in a medical cart comprising
providing the medical products with identification tags;
storing the medical products in designated compartments in the medical cart;
sensing the identification tags in each designated compartment to determine the presence of required medical products in each designated compartment; and
providing an output indicating whether the required medical products are stored in the medical cart.
